(19) United States
(12) Patent Application Publication
(10) Pub. No.: US 2007/0148247 A1
(43) Pub. Date: Jun. 28, 2007

(54) Pharmaceutical composition which reduces or eliminates drug abuse potential

(76) Inventors: Yatindra Joshi, Princeton, NJ (US); Russell Somma, Sparta, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/705,658

(22) Filed: Feb. 13, 2007

Related U.S. Application Data
(63) Continuation of application No. 10/656,546, filed on Sep. 5, 2003, now abandoned.
Application No. 10/656,546 is a continuation of application No. 09/942,808, filed on Aug. 30, 2001, now abandoned.
(60) Provisional application No. 60/287,509, filed on Apr. 30, 2001.

(51) Int. Cl. A61K 31/137; A61K 9/14

(52) U.S. Cl. 424/486; 424/488; 514/649

(57) ABSTRACT

A pharmaceutical composition which reduces or eliminates the drug abuse potential of central nervous system stimulant comprising: (a) a drug selected from the group consisting of methylphenidate, amphetamine, methamphetamine, and combinations thereof; and (b) a gel forming polymer wherein the gel forming polymer is a polymer that forms a gel when contacted with moisture or placed in an aqueous solution. The present invention is based on the discovery that a central nervous system stimulant such as methylphenidate in combination with a gel forming polymer reduces or eliminates drug abuse potential by swelling in the presence of moisture, and thus, preventing nasal absorption and injectability of the drug.

### FIELD OF THE INVENTION

The present invention relates to a pharmaceutical composition which reduces or eliminates drug abuse potential. More specifically, the composition comprises a central nervous system stimulant and a gel forming polymer.

### BACKGROUND OF THE INVENTION

Methylphenidate, which is commercially available under the trademark Ritalin® from Novartis Pharmaceuticals Corporation, is a central nervous system stimulant. Other examples of central nervous stimulants are amphetamine and methaniphetamine. Central nervous stimulants activate the brain stem arousal system to effect stimulation of the patient.

Methylphenidate is the most commonly prescribed psychotropic medication for children in the United States, primarily for the treatment of children diagnosed with attention deficit disorder (ADD) and Attention Deficit Hyperactivity Disorder (ADHD), and thus, is widely available. In addition, methylphenidate has been found to be particularly useful for treating Acquired Immunodeficiency Syndrome (AIDS) patients who suffer from cognitive decline. See Navia et al., Annals of Neurology, 19:517-524 (1986).

Methylphenidate is described in U.S. Pat. Nos. 2,838,519 and 2,957,880. U.S. Pat. Nos. 5,922,736; 5,908,850; 5,773,478; 6,113,879 describe administering d-threo methylphenidate to treat nervous system disorders. U.S. Pat. Nos. 5,936,091 and 5,965,734 describe processes and intermediates for preparing 2-substituted d-threo piperidines. U.S. Pat. Nos. 6,100,401; 6,121,453; and 6,162,919 describe processes for preparing substantially the single enantiomer d-threo methylphenidate. U.S. Pat. Nos. 5,874,090 and 5,837,284 describe sustained release formulations of methylphenidate.

In addition to their important medical uses, central nervous system stimulants are employed commonly, by such means as inhalation and intravenously, for illicit purposes, including emotional, psychological, euphoric, hallucinogenic, and psychedelic experiences. These purposes and the physical dependence accompanying the administration of these drugs has led to drug abuse. Drug abuse has become for many habituates a way of life. To a rapidly growing segment of the world population, use of these drugs is often seen as fashionable.

WO 97/33566 describes an opioid composition which has a low potential for abuse. This is achieved by incorporating an opioid antagonist in the composition in an amount to reduce the effect of the opioid. Examples of opioid antagonists disclosed in WO 97/33566 are naltrexone, naloxone, nalmefene, nalide, nalmexone, nalorphine, nalpuphine, nalorphine, and dinicotinate.

While central nervous stimulants are a necessary part of modern medicine, it would be highly desirable to provide a pharmaceutical composition comprising a central nervous stimulant which reduces or eliminates drug abuse potential without decreasing the effectiveness of the drug.

### SUMMARY OF THE INVENTION

The present invention relates to a pharmaceutical composition which reduces or eliminates the drug abuse potential of central nervous system stimulant comprising: (a) a drug selected from the group consisting of methylphenidate, amphetamine, methamphetamine, and combinations thereof; and (b) a gel forming polymer wherein the gel forming polymer is a polymer that forms a gel when contacted with moisture or placed in an aqueous solution.

The present invention is based on the discovery that a central nervous system stimulant such as methylphenidate in combination with gel forming polymer reduces or eliminates potential drug abuse by swelling in the presence of moisture which is, for example, present in the dermis layer of skin and mucous membrane, and thus, prevents nasal absorption and injectability of the drug.

### DESCRIPTION OF THE INVENTION

The invention is directed to a pharmaceutical composition which reduces or eliminates the drug abuse potential of central nervous system stimulant. The composition comprises a central nervous system stimulant and a gel forming polymer. Component (a) of the composition of the invention is a central nervous system stimulant such as methylphenidate, amphetamine, and methamphetamine. Pharmaceutically acceptable salt forms of the central nervous system stimulant are included within the term “central nervous system stimulant”. A combination of central nervous system stimulants may also be used.

As used herein, “methylphenidate” includes the following four optical isomers: d-threo-methylphenidate, l-threo-methylphenidate, d-erythro-methylphenidate, and l-erythro-methylphenidate. A preferred isomer is d-threo-methylphenidate. A combination of isomers may also be used, for example, dl-threo-methylphenidate. Most preferably, the methylphenidate is methylphenidate hydrochloride.

The effective dosage for the central nervous system stimulant may vary depending on the concentration of the drug, the mode of administration, the condition being treated, and the severity of the condition being treated. In addition, the effective dosage depends on a variety of factors which are specific to the patient being treated, such as species type, age, weight, and sex.

In a preferred embodiment of the invention, the amount of central nervous system stimulant in the compositions of the invention is from about 0.1 to about 90 weight percent, more preferably from about 1 to about 50 weight percent, based on the total weight of the composition. Most preferably, the amount of central nervous system stimulant in the compositions is from about 2 to about 10 weight percent, based on the total weight of the composition.

Component (b) of the composition of the invention is a gel forming polymer. The gel forming polymer is any polymer that forms a gel when contacted with moisture or placed in an aqueous solution. The gel forming polymers may be used alone or in combination with other gel forming polymers. The gel forming polymers include natural and synthetic polymers, and may be crosslinked or not crosslinked. Examples of gel forming polymers include, but are not limited to, the following:

(a) Polysaccharide such as agar, carrageenan, modified cellulose, and starch. Preferred carrageenans are GELCARIN GP911 and GELCARIN 379, which are available from FMC Corporation. Preferred modified celluloses are hydroxyethylcellulose, hydroxypropylmethylcellulose, sodium carboxymethyl cellulose, hydroxypropyl methyl cellulose phthalate or acetate succinate, cellulose acetate phthalate, methyl cellulose phthalate, and microcrystalline cellulose. Preferred starches are cold water swelling starches such as starches sold by National Starch under the trademarks NOVATION, ULTRA-SPERSE, and ULTRA-TEX, and sodium carboxymethyl starch, and starch acetate phthalate.

(b) Gelatin. Preferred gelatins are GELATIN G 9382 and GELATIN G 2625, which are available from Sigma Chemicals.

(c) Polyglucosamine or its various chemically modified variants. Preferred polyglucosamines are SEACURE 343 and SEACURE 443, which are available from Pronova Biopolymers. These materials form a gel at a pH of 5 to 7.

(d) Hydrophilic colloid such as derivatives of alginic acid. Preferred derivatives of alginic acid are calcium alginate, sodium alginate, potassium alginate, and propylene glycol alginate.

(e) Crosslinkable hydrophilic polymer. Preferred crosslinkable hydrophilic polymers are polyvinyl pyrrolidone, carboxymethylamide, potassium methacrylatedivinylbenzene copolymer, polyvinylalcohol, polyoxyethyleneglycol, polyethylene glycol, carboxypolymethylene, polymers and copolymers of acrylic acid and/or methacrylic acid and/or their esters (for example ACRYSOL and ACULYN, available from Rohm & Haas, and a homopolymer of acrylic acid crosslinked with allyl sucrose or allylpentaerythritol, and a copolymer of acrylic acid and an alkyl acrylate and crosslinked with allylpentaerythritol, wherein the alkyl group has from 10 to 30 carbon atoms, for example CARBOPOL, available from B. F. Goodrich), polyvinyl pyrrolidone/acrylic acid (for example ACRYLIDONE Anionic Copolymer 1033 or 1042, available from ISP), polymethyl vinyl ether/maleic anhydride (for example GANTREZ AN Copolymer S-97, available from GAF), polyethylene/maleic anhydride, polymethyl methacrylate, polyethyl methacrylate, polybutyl methacylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctadecyl acrylate, copolymer of acrylic and methacrylic acid ester with a lower ammonium group content (for example EUDRAGIT RS, available from Rohm & Haas), copolymer of acrylic and methacrylic acid ester and trimethyl ammonium methacrylate (for example EUDRAGIT RL, available from Rohm & Haas), polyvinyl acetate; polyvinyl acetate phthalate, maleic acid anhydride-vinyl methyl ether, styrene-maleic acid, 2-ethyl-hexyl-acrylate maleic acid anhydride, crotonic acid-vinyl acetate, glutaminic acidiglutamic acid ester, polyarginine, polyethylene, polypropylene, polyethylene oxide (for example POLYOX, available from Union Carbide), polyethylene terephthalate, polyvinyl isobutyl ether, polyvinyl chloride, polyurethane, and vinyl pyrrolidone/dimethylamino ethyl methacrylate, (for example GAFQUAT 755, available from GAF).

(f) An acrylate ester polymerized with a monomer selected from a vinyl-substituted heterocyclic compound containing at least one of a nitrogen or a sulfur atom, (meth)acrylamide, a mono- or di-C₁-C₄ alkylamino C₁-C₄ alkyl (meth)acrylate, or a mono or di-C,-C₄ alkylamino C₁-C₄ alkyl acrylamide. Specific examples of such monomers are N,N-dimethylamino ethyl methacrylate, N,N-diethylamino ethyl acrylate, N,N-diethylamino ethyl methacrylate, N-t-butylamino ethyl acrylate, N-t-butylamino ethyl methacrylate, N,N-dimethylamino propyl acrylamide, N,N-dimethylamino propyl methacrylamide, N,N-diethylamino propyl acrylamide, and N,N-diethylamino propyl methacrylamide.

In a preferred embodiment the gel forming polymer is selected from polyethylene oxide, sodium alginate, a homopolymer of acrylic acid crosslinked with allyl sucrose or allylpentaerythritol, and a copolymer of acrylic acid and an alkyl acrylate and crosslinked with allylpentaerythritol, wherein the alkyl group has from 10 to 30 carbon atoms.

The gel forming polymer preferably has a molecular weight of from about 70,000 to about 2,000,000. More preferably, the molecular weight of the gel forming polymer is from about 100,000 to about 1,000,000.

The amount of gel forming polymer in the compositions of the invention is preferably from about 2 to 40 weight percent, based on the total weight of the composition. More preferably, the amount of gel forming polymer is from about 5 to about 30 weight percent, more preferably from about 10 to about 20 weight percent.

The pH range of the gel forming polymers is preferably between about 5.5 and 8.5. A base such as sodium or calcium hydroxide can be added to increase the pH to the desired range. Similarly, buffers such as calcium carbonate, diethyl carbonate, diphenyl carbonate, and sodium citrate, may be added to control the pH.

Conventional methods of preparing the gel forming polymers in the various forms are known by those skilled in the art. Such methods include solution polymerization, precipitation polymerization and emulsion polymerization.

Additional ingredients which may be used in the compositions of the invention include natural and/or artificial ingredients which are commonly used to prepare oral pharmaceutical dosage forms. Examples of additional ingredients include enteric coating agents, diluents, binders, humectants, disintegrants, anti caking agents, amino acids, fibers, solubilizers, emulsifiers, flavorants, sweeteners, enzymes, fillers, buffers, stabilizers, colorants, dyes, plasticizing agents, antioxidants, anti-adherents, preservatives, electrolytes, glidants, lubricants, and carrier materials. A combination of additional ingredients may also be used. Such ingredients are known to those skilled in the art, and thus, only a limited number will be specifically referenced. Preferably the additional ingredients are used in the compositions of the invention in an amount that corresponds to an amount generally recognized as safe (GRAS) and effective by the United States Food and Drug Administration, the Environmental Protection Agency, the United States Department of Agriculture, or other comparable regulatory agency. For those additional ingredients for which no regulatory approval has been obtained, then an amount generally accepted in the art as both safe and efficacious is preferred.

Examples of humectants that can be used in the compositions of the invention include but are not limited to: sucrose, sorbitol, glycerol, propylene glycol, poly-(ethylene glycol), N-methyl pyrrolidone, N-ethyl pyrrolidone, diacetone alcohol, .gamma.-butyryl lactone, ethyl lactate, low molecular weight polyethylene glycol, or combinations thereof.

Examples of glidants that can be used in the compositions of the invention include but are not limited to: silica, magnesium trisilicate, powdered cellulose, starch, and talc. Colloidal silica and colloidal silicone dioxide are particularly preferred.

Examples of fillers that can be used in the compositions of the invention include but are not limited to: confectioner's sugar, compressible sugar, dextrates, dextrin, dextrose, lactose, sucrose, mannitol, microcrystalline cellulose, powdered cellulose, sorbitol, and tribasic calcium phosphate.

Examples of lubricants that can be used in the compositions of the invention include but are not limited to: stearic acids and its salts such as Mg, Al or Ca stearate, polyethylene glycol 4000-8000, talc, sodium benzoate, sodium acetate, leucine, sodium oleate, sodium lauryl sulfate, and magnesium lauryl sulfate.

Examples of solubilizers and/or emulsifiers that can be used in the compositions of the invention include but are not limited to: sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20.

Examples of antioxidants that can be used in the compositions of the invention include but are not limited to: sodium sulphite, sodium hydrogen sulphite, sodium metabisulphite, ascorbic acid, ascorbylpalmitate, -myristate, -stearate, gallic acid, gallic acid alkyl ester, butylhydroxyamisol, nordihydroguaiaretic acid, tocopherols as well as synergists (substances which bind heavy metals through complex formation, for example lecithin, ascorbic acid, phosphoric acid ethylene diamine tetracetic acid, citrates, tartrates). Addition of synergists substantially increases the antioxygenic effect of the antioxidants.

Examples of preservatives that can be used in the compositions of the invention include but are not limited to: sorbic acid, p-hydroxybenzoic acid esters, benzoic acid, sodium benzoate, trichloroisobutyl alcohol, phenol, cresol, benzethonium chloride, chlorhexidine and formalin derivatives.

The total amount of additional ingredients in the compositions of the invention are preferably from about 30 to about 75 weight percent, based on the total weight of the composition. More preferably, the total amount of additional ingredients is from about 50 to about 70 weight percent, most preferably from about 53 to about 67 weight percent, based on the total weight of the composition.

The following examples further describe the materials and methods used in carrying out the invention. The examples are not intended to limit the invention in any manner.

### EXAMPLE 1

Preparation of Chewable Tablets Containing 2.5% dl-Methylphenidate and 10% Gel Forming Polymer.

Composition

| | | |
|---|---|---|
| dl-methylphenidate | 5.0 | gm |
| POLYOX ® | 20.0 | gm |
| lactose | 75.0 | gm |
| talc | 3.0 | gm |
| mannitol | 90.0 | gm |
| stearic acid | 2.0 | gm |
| 5% gelatin solution in demineralized water | 4.0 | gm |
| saccharin | 1.0 | gm |

All the solid ingredients are first forced through a sieve of 0.25 mm mesh width. The mannitoli dl-methylphenidate, and lactose are mixed, granulated with the addition of gelatin solution, forced through a sieve of 2 mm mesh width, dried at 50° C. and again forced through a sieve of 1.7 mm mesh width. POLYOX®, talc and saccharin are added to the dried mixture of drug substance. The stearic acid is added and the final blend is made. The resulting blend is compressed to form 7 mm round standard concave tablets.

### EXAMPLE 2

Preparation of Tablets Containing 4% d-Methylphenidate and 1.2% Gel Forming Polymer.

Composition

| | | |
|---|---|---|
| d-methylphenidate | 10.0 | gm |
| PEG 8000 | 3.0 | gm |
| sucrose | 3.0 | gm |
| starch | 20.0 | gm |
| lactose | 170 | gm |
| talc | 2.0 | gm |
| magnesium stearate | 2.0 | gm |
| sodium alginate | 40.0 | gm |
| demineralized water | | |

All the solid ingredients are first forced through a sieve of 0.6 mm mesh width. The dl-methylphenidate, a portion of the lactose, starch, and sucrose are mixed then granulated with the PEG 8000 solution. The granulation is dried overnight at 50° C., and then forced through a sieve of 1.2 mm mesh width. The remaining lactose, talc, magnesium stearate and sodium alginate are blended with the dried material. The resulting blend is compressed to form 8 mm round standard concave tablets.

### EXAMPLE 3

Preparation of Capsules Containing 8% dl-Methylphenidate and 20% Gel Forming Polymer.

Composition (for 1000 tablets)

| | | |
|---|---|---|
| dl-methylphenidate | 20.0 | gm |
| microcrystalline cellulose | 88.0 | gm |
| modified starch | 88.0 | gm |
| magnesium stearate | 4.0 | gm |
| CARBOPOL ® | 50.0 | gm |

The microcrystalline cellulose, modified starch, and di-methylphenidate are granulated with water and then passed through a 0.9 mm mesh screen and dried at 50° C. The dried material is passed through a 0.9 mm mesh screen and blended with the magnesium stearate and CARBOPOL®. The resulting blend is encapsulated using size #1 hard shell gelatin capsule.

### EXAMPLE 4

Study of Swelling Activity

A tablet prepared in Example 1 is placed on a glass plate and crushed to form a powder. The powder is added to 1 ml of water and stirred for one minute. Gel formation occurs.

### EXAMPLE 5

Study of Swelling Activity

A tablet prepared in Example 2 is placed on a glass plate and crushed to form a powder. The powder is added to 1 ml of water and stirred for one minute. Gel formation occurs.

### EXAMPLE 6

Study of Swelling Activity

A capsule prepared in Example 3 is placed on a glass plate and crushed. The material is combined with 1 ml of water. Gel formation occurs.

The present invention is based on the discovery that a central nervous system stimulant such as methylphenidate in combination with a gel forming polymer reduces or eliminates potential drug abuse by swelling in the presence of moisture which is, for example, present in the dermis layer of skin and mucous membrane, and thus, preventing nasal absorption and injectability of the drug.

While the invention has been described with particular reference to certain embodiments thereof, it will be understood that changes and modifications may be made by those of ordinary skill within the scope and spirit of the following claims:

1-23. (canceled)

24. A pharmaceutical composition which reduces or eliminates the drug abuse potential of central nervous system stimulant comprising:
a) a drug selected from the group consisting of methylphenidate, amphetamine, methamphetamine, and combinations thereof; and
b) a gel forming polymer wherein the gel forming polymer is a polymer that forms a gel when contacted with moisture or placed in an aqueous solution.

25. The composition according to claim 24 wherein the gel forming polymer is selected from the group consisting of a polysaccharide, gelatin, polyglucosamine, hydrophilic colloid, cross-linkable hydrophilic polymer, an acrylate ester polymerized with a monomer selected from the group consisting of a vinyl-substituted heterocyclic compound containing at least one of a nitrogen or a sulfur atom, (meth)acrylamide, a mono- or di-C₁-C₄ alkylamino C₁-C₄ alkyl (meth)acrylate, and a mono or di-C₁-C₄ alkylamino C₁-C₄ alkyl acrylamide and combinations thereof.

26. The composition according to claim 25 wherein the polysaccharide is selected from the group consisting of an agar, carrageenan, modified cellulose and starch.

27. The composition according to claim 26 wherein the polysaccharide is selected from the group consisting of hydroxyethylcellulose, hydroxypropylmethylcellulose, sodium carboxymethyl cellulose, hydroxypropyl methyl cellulose phthalate or acetate succinate, cellulose acetate phthalate, methyl cellulose phthalate, microcrystalline cellulose, a cold water swelling starch, sodium carboxymethyl starch and starch acetate phthalate.

28. The composition according to claim 25 wherein the hydrophilic colloid is a derivative of alginic acid.

29. The composition according to claim 28 wherein the derivative of alginic acid is selected from the group consisting of calcium alginate, sodium alginate, potassium alginate and propylene glycol alginate.

30. The composition according to claim 25 wherein the cross-linkable hydrophilic polymer is selected from the group consisting of polyvinyl pyrrolidone, carboxymethylamide, potassium methacrylatedivinylbenzene, polyvinylalcohol, polyoxyethyleneglycol, polyethylene glycol, carboxypolymethylene, polyacrylic acid, polymethacrylic acid, polyvinyl pyrrolidone/acrylic acid, polymethyl vinyl ether/maleic anhydride, polyethylene/maleic anhydride, polymethyl methacrylate, polyethyl methacrylate, polybutyl methacylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctadecyl acrylate, copolymer of acrylic and methacrylic acid ester with a lower ammonium group content, copolymer of acrylic and methacrylic acid ester and trimethyl ammonium methacrylate, polyvinyl acetate, polyvinyl acetate phthalate, maleic acid anhydride-vinyl methyl ether, styrene-maleic acid, 2-ethyl-hexyl-acrylate maleic acid anhydride, crotonic acid-vinyl acetate, glutaminic acid/glutamic acid ester, polyarginine, polyethylene, polypropylene, polyethylene oxide, polyethylene terephthalate, polyvinyl isobutyl ether, polyvinyl chloride, polyurethane and vinyl pyrrolidone/dimethylamino ethyl methacrylate.

31. The composition according to claim 25 wherein the acrylate ester is polymerized with a monomer selected from the group consisting of N,N-dimethylamino ethyl methacrylate, N,N-diethylamino ethyl acrylate, N,N-diethylamino ethyl methacrylate, N-t-butylamino ethyl acrylate, N-t-butylamino ethyl methacrylate, N,N-dimethylamino propyl acrylamide, N,N-dimethylamino propyl methacrylamide, N,N-diethylamino propyl acrylamide, and N,N-diethylamino propyl methacrylamide.

32. The composition according to claim 25 wherein the gel forming polymer is selected from the group consisting of polyethylene oxide, sodium alginate, a homopolymer of acrylic acid cross-linked with allyl sucrose or allylpentaerythritol, and a copolymer of acrylic acid and an alkyl acrylate and cross-linked with allylpentaerythritol, wherein the alkyl group has from 10-30 carbon atoms.

33. The composition according to claim 24 wherein the gel forming polymer has a molecular weight of from about 70,000 to about 2,000,000.

34. The composition according to claim 24 which additionally comprises a pH modifier.

35. The composition according to claim 34 wherein the pH modifier is selected from the group consisting of sodium hydroxide, calcium hydroxide, calcium carbonate, diethyl carbonate, diphenyl carbonate and combinations thereof.

36. The composition according to claim 24 wherein the gel forming polymer is present in an amount of from about 2 to about 40 weight percent, based on the total weight of the composition.

37. The composition according to claim 24 wherein the central nervous system stimulant is present in an amount of from about 0.1 to about 90 weight percent, based on the total weight of the composition.

38. The composition according to claim 24 which is in a form selected from the group consisting of powder, granules, solution, suspension, emulsion and combinations thereof.

39. A method for the reduction or elimination of the drug abuse potential of central nervous system stimulants by subjects in need of central nervous system stimulation therapy by providing said subjects with a composition comprising
(a) a drug selected from the group consisting of methylphenidate, amphetamine, methamphetamine, and combinations thereof; and
(b) a gel forming polymer wherein the gel forming polymer is a polymer that forms a gel when contacted with moisture or placed in an aqueous solution.
